(19) United States
(12) Patent Application Publication
(10) Pub. No.: US 2020/0121941 A1
(43) Pub. Date: Apr. 23, 2020

(54) SKIN ATTACHMENT COMPRISING LIGHT-EMITTING ELEMENT AND STORAGE APPARATUS THEREFOR

(71) Applicant: NEXPRESS INC., Seongnam-si, Gyeonggi-do (KR)

(72) Inventors: Young Woo KWON, Miryang-si, Gyeongsangnam-do (KR); Seok Hee KANG, Busan (KR); Sung Jae HWANG, Seoul (KR)

(73) Assignee: NEXPRESS INC., Seongnam-si, Gyeonggi-do (KR)

(21) Appl. No.: 16/084,005

(22) PCT Filed: Mar. 13, 2017
(86) PCT No.: PCT/KR2017/002691

§ 371 Date: Nov. 8, 2018

(30) Foreign Application Priority Data
Mar. 11, 2016 (KR) 10-2016-0029603
Jul. 7, 2016 (KR) 10-2016-0086116

(51) Int. Cl. A61N 5/06; A61B 5/0205; A61F 7/00; A61M 37/00

(52) U.S. Cl. CPC A61N 5/062; A61N 5/0616; A61B 5/02055; A61F 7/007; A61B 5/02438; A61N 2005/0663; A61N 2005/0659; A61N 2005/0626; A61M 37/00

(57) ABSTRACT

The present invention relates to a skin attachment comprising light emitting elements and a storage apparatus therefor. According to one aspect of the invention, there is provided a skin attachment, comprising: an attachment unit attached to skin of a user, wherein the attachment unit is capable of being bent and attached along a curved surface of the skin of the user; a light emitting unit disposed in the attachment unit, wherein the light emitting unit comprises at least one light emitting element; a diffusion unit configured to diffuse light from the light emitting unit, wherein the diffusion unit comprises scattering particles or microlenses; and a control unit disposed in the attachment unit and configured to control the light emitting unit.

### CROSS-REFERENCE TO RELATED APPLICATION(S)

This application a continuation application of Patent Cooperation Treaty (PCI) International Application Serial No. PCT/KP2017/002691, filed on Mar. 13, 2017, which claims priority to Korean Patent application Serial Nos. 10-2016-0029603 and 10-2016-0086116, filed on March 11 and Jul. 7, 2016. The entire contents of PCI International Application Serial No. PCT/KR2017/002691 and Korean Patent Application Serial Nos. 10-2016-0029603 and 10-2016-0086116 are hereby incorporated by reference.

### FIELD OF THE INVENTION

The present invention relates to a skin attachment comprising light emitting elements and a storage apparatus therefor.

### BACKGROUND

People's desires for skin care are growing. In other words, most people desire clean skin, bright skin, skin with no or less symptoms such as inflammation, and the like. Accordingly, a number of solutions are offered, such as dermatologic care or treatment, patch type products, sheet mask type products, injection medicines, ointments, and the like. However, each of them has some disadvantages in terms of cost and effectiveness.

Phototherapy is a relatively recently known measure. The phototherapy literally means emitting light onto a specific region of skin for treatment of inflammation, elimination of infiammagens, reduction of aging rates, improvement of wrinkles, assistance for regeneration process, support for absorption of medicines or the like, application of heat, and the like. However, the phototherapy has not been actually popularized due to the significant lack of user friendliness of the apparatuses or the like used therefor.

In this regard, the present inventor(s) suggest a novel skin attachment co rising light emitting elements for phototherapy.

Further, the present inventor(s) also suggest a storage apparatus that stores the skin attachment and provides various functions.

### SUMMARY OF THE INVENTION

One object of the present invention is to provide a skin attachment comprising light emitting elements.

Another object of the invention is to provide a user-friendly skin attachment for implementing phototherapy for skin.

Yet another object of the invention is to provide a skin attachment for acquiring various information on skin.

Still another object of the invention is to implement phototherapy in which feedback control is feasible.

Still yet another object of the invention is to provide a storage apparatus that stores a skin attachment comprising light emitting elements and provides various functions.

The representative configurations of the invention to achieve the above objects are described below.

According to one aspect of the invention, there is provided a skin attachment, comprising: an attachment unit attached to skin of a user, wherein the attachment unit is capable of being bent and attached along a curved surface of the skin of the user; a light emitting unit disposed in the attachment unit, wherein the light emitting unit comprises at least one light emitting element; a diffusion unit configured to diffuse light from the light emitting unit, wherein the diffusion unit comprises scattering particles or microlenses; and a control unit disposed in the attachment unit and configured to control the light emitting unit.

According to another aspect of the invention, there is provided an assembly comprising a skin attachment and a storage apparatus, wherein the skin attachment comprises: an attachment unit attached to skin of a user, wherein the attachment unit is capable of being bent and attached along a curved surface of the skin of the user; a light emitting unit disposed in the attachment unit, wherein the light emitting unit comprises at least one light emitting element; and a control unit disposed in the attachment unit and configured to control the light emitting unit, and wherein the storage apparatus comprises: a housing unit configured to house the skin attachment; and a charging unit configured to charge the skin attachment.

In addition, there may be further provided other configurations according to the technical idea of the invention.

According to the invention, there is provided a skin attachment comprising light emitting elements.

According to the invention, there is provided a user-friendly skin attachment for implementing phototherapy for skin.

According to the invention, there is provided a skin attachment for acquiring various information on skin.

According to the invention, phototherapy is implemented in which feedback control is feasible.

According to the invention, there is provided a storage apparatus that stores a skin attachment comprising light emitting elements and provides various functions.

### BRIEF DESCRIPTION OF THE DRAWINGS

FIG. 1 schematically shows the configuration of a skin attachment comprising light emitting elements according to one embodiment of the invention.

FIG. 2 shows that a user wears a neck band according to one embodiment of the invention.

FIG. 3 conceptually shows various types of microneedles.

FIG. 4 conceptually shows a collection of skin attachment patches according to one embodiment of the invention.

FIG. 5 conceptually shows a skin attachment in the form of a sheet mask according to one embodiment of the invention.

FIG. 6 conceptually shows an additional layer that may be coupled to and used in conjunction with a skin attachment according to one embodiment of the invention.

FIG. 7 conceptually shows a diffusion unit that may be included in a skin attachment according to one embodiment of the invention.

FIG. 8 illustratively shows a situation in which display is performed through light emitting elements of a skin attachment according to one embodiment of the invention.

FIG. 9 schematically shows a skin attachment storage apparatus according to one embodiment of the invention.

FIG. 10 schematically shows a skin attachment storage apparatus according to one embodiment of the invention.

FIG. 11 schematically shows a skin attachment storage apparatus according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the inventiont is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

### Skin Attachment

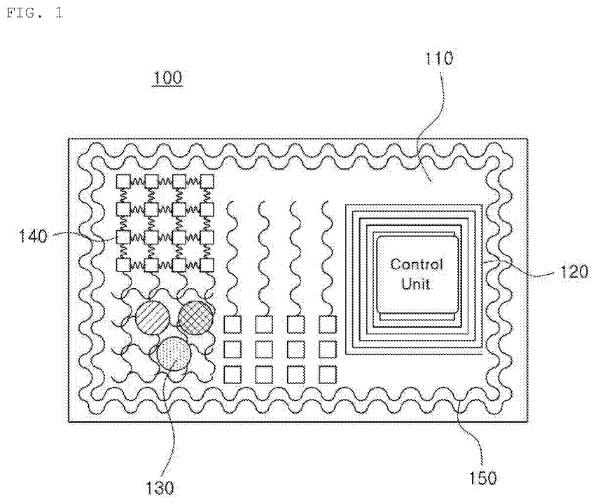

FIG. 1 schematically shows the configuration of a skin attachment comprising tight emitting elements according to one embodiment of the invention.

Referring to FIG. 1, a skin attachment 100 may comprise at least some of an attachment unit 110 that is directly attached to skin; a control unit 120 configured to control at least some of the components to be described below; a light emitting unit 130 comprising at least one light emitting element; a power supply unit 140 configured to supply power to the control unit 120, the light emitting unit 130, and the like; and microwire 150 configured to receive power from the outside of the skin attachment 100. At least some of the control unit 120, the light emitting unit 130, the power supply unit 140, and the microwire 150 may be disposed in the attachment unit 110. In this case, known FPCB (Flexible Printed Circuit Board) techniques or electronic printing techniques may be employed.

First, the attachment unit 110 according to one embodiment of the invention will be described. One side of the attachment unit 110 is attached or adhered to skin or the like of a user, and an adhesive material may be applied to the side, as necessary. In addition, cosmetic substances for skin care or medicinal substances for skin treatment or skin health improvement may be further applied to the attachment unit 110. The attachment unit 110 may be composed of fibers, hydrocolloids, collagen, kinesio bands, plastic films, and the like. Accordingly, the skin attachment 100 and the attachment unit 110 serving as the body of the skin attachment 100 may be flexible enough to be bent and attached along a curved surface of the skin.

At least a part of the attachment unit 110 may include a conductive grid (not shown) that is elastic and bendable. The conductive grid may be intended to send electric currents or transmit electrical signals between the components disposed in the attachment unit 110. The conductive grid may also serve as an antenna for wireless charging to be described below. The conductive grid may be composed of at least one of metal, metal nanowire, conductive ink material, liquid metal, carbon nanotube, conductive graphene, conductive polymer material, and the like. The conductive grid may be disposed on or adjacent to a surface opposite to the side of the attachment unit 110 that is attached to the skin.

Meanwhile, at least a part of the attachment unit 110 may include an ionizing material or electrolyte. The ionizing material or electrolyte may be contained in the attachment unit 110 as encapsulated in a capsule (not shown), or may be injected into the attachment unit 110 by a syringe or the like at a later time (e.g., immediately before the attachment unit 110 is used by the user). In the former case, when the capsule is blown by an external impact or an external pointed object (e.g., needle) to discharge the ionizing material or electrolyte to the attachment unit 110, generation of electricity may be achieved via the conductive grid. In the latter case, the ionizing material or electrolyte may start to act for generation of electricity as soon as it is injected. The generation of electricity may result from an oxidation-reduction reaction via the conductive grid. Thus, the conductive grid may be composed of two or more types of metal. Electricity generated as above may be obtained as a power source by the power supply unit 140, or may be directly used by the light emitting unit 130 or the like.

The attachment unit 110 may be in the form of a patch or band that may be attached to a small region of the user's skin, a full-size sheet mask sized similarly to the user's face, or a bandage that may be applied to a body part of the user, depending on the use of the skin attachment 100.

Next, the control unit 120 according to one embodiment of the invention will be described. The control unit 120 is an ultra-thin processor that may control switching on/off or light intensity of each of at least one light emitting element of the Light emitting unit 130, power supply of the power supply unit 140, switching on/off of a sensor unit to be described below, and the like. In some cases, some of the controls of the control unit 120 may be performed by an external device (not shown) capable of wired or wireless communication (e.g., WiFi or Bluetooth communication) with the control unit 120. The external device may be a consumer electronic appliance of the user or another person, such as a computer, a smart phone, and a smart pad.

In some cases, the skin attachment 100 may further comprise a sensor unit (not shown). The sensor unit may include at least one sensor (not shown) to measure temperature, pulse, oxygen saturation, and the like in the user's skin. The values of the above skin indexes may be transmitted to the control unit 120 by the sensor unit. Accordingly, the control unit 120 may transmit, to the external device, the measurement values related to the skin of the body part to which the skin attachment 100 is attached. The external device may accordingly command a predetermined feedback control on the basis of the received measurement values.

Meanwhile, the sensor unit may sense more various values in addition to the values of the above skin indexes. For example, the sensor unit may further include at least one of a gyroscope sensor, an image sensor, a capacitance sensor, an impedance sensing electrode, and the like. The corresponding measurement values may also be transmitted to the control unit 120 or the external device. The external device may also command a predetermined feedback control on the basis of the received measurement values.

A few examples the feedback control will be described below.

For example, when the user applies heat to his/her skin by means of the skin attachment 100, a temperature sensor of the sensor unit may periodically or intermittently check the temperature of the corresponding region and notify the control unit 120 or the external device of the temperature. On the basis of this, the control unit 120 or the external device may actively stop the application of heat by determining an appropriate time to stop the heat application.

As another example, when the user is using the skin attachment 100, a gyroscope sensor may periodically or intermittently check information on the user's posture and notify the control unit 120 or the external device of the information. On the basis of this, the control unit 120 or the external device may actively start or stop phototherapy or the like at a point of time when it is determined that the user is asleep, or after a predetermined period of time has elapsed from the point of time. Meanwhile, the details of the phototherapy (e.g., the light intensity of a specific light emitting element or the type of light emitting element that emits light) may be changed according to the information on the posture. On the other hand, the gyroscope sensor may enable the control unit 120 or the external device to determine whether the user is using the skin attachment 100 or to determine the start/end time of the use.

As yet another example, when the user is using the skin attachment 100, an image sensor may periodically or intermittently check visual information on the user's skin and notify the control unit 120 or the external device of the information. On the basis of this, the control unit 120 the external device may change the details of phototherapy (e.g., the light intensity of a specific light emitting element or the type of light emitting element that emits light) or determine whether the phototherapy is indeed appropriate, according to a color of the user's skin or a change in the color, a degree to which the use's skin is exposed to ultraviolet light, or the like.

As still another example, a capacitance sensor may measure the capacitance of each point between the user's skin and the skin attachment 100 and notify the control unit 120 or the external device of the capacitance. On the basis of this, the control unit 120 or the external device may easily recognize the degree of flexure of the user's skin, and may accordingly change the details of phototherapy.

As still yet another example, an impedance sensing electrode may measure the impedance of each region of the user's skin and notify the control unit 120 or the external device of information on water or oil of the user s skin. On the basis of this, the control unit 120 or the external device may easily recognize the content, distribution, or the like of the water or oil of the user's skin, and may accordingly change the details of phototherapy.

Meanwhile, such an electrode may not only serve as a sensor but may also serve to flow a microcurrent or an electromagnetic wave to the user's skin to apply slight heat. The slight heat stimulates a dermis muscle layer to help absorption of cosmetic or medicinal substances, collagen synthesis, and the like in the user's skin.

Next, the light emitting unit 130 according to one embodiment of the invention will be described. The light emitting unit 130 may comprise at least one light emitting element. The light emitting element may be a light emitting diode (LED) or other light, source capable of phototherapy. Each of the at least one light emitting element may emit visible light or infrared light (particularly, near-infrared light) of a specific wavelength band. Here, the wavelength bands of the light emitting elements may be the same or different.

Alternatively, a part of the light emitting elements (i.e., one set of light emitting elements) may have a first wavelength band, and another part of the light emitting elements (i.e., another set of light emitting elements) may have a second wavelength band. In this case, a set of light emitting elements having a third or another wavelength band may also be assumed. In order to allow light to be emitted at a more uniform intensity, the light emitting elements of a specific wavelength band may be disposed in the light emitting unit 130 at a predetermined distance from each other.

According to the results of existing clinical studies conducted by scholars, light of a wavelength band of 400 to 430 nanometers may be absorbed by epithelial tissue of skin. Since it stimulates porphyrin in the skin and destroys bacteria, it is well suited for symptom improvement or treatment of acne, which is mainly caused by bacteria.

Meanwhile, light of a wavelength band of 630 to 680 nanometers penetrates into dermal tissue of skin to stimulate mitochondria within the skin and activate the production of ATP, which may be significantly beneficial to invigoration of cell activity and improvement of inflammation. This may lead to cell conduction, surface circulation, anti-inflammatory release, and the like.

Further, it is known that lights of a wavelength band of 800 to 900 nanometers (i.e., near-infrared light) has the effect of relieving the pain caused by skin diseases, and may mildly heat skin to help absorption of cosmetic or medicinal substances into the skin.

Accordingly, the light emitting unit 130 may comprise at least one light emitting element that emits light of a specific wavelength band required for a specific use. Switching on/off or light intensity of each of the at least one light emitting element may be controlled by the control unit 120. However, in some cases, each light emitting element of the light emitting unit 130 may naturally be switched on as the above-described electrolyte or the like permeates in the attachment unit 110.

Next, the power supply unit 140 according to one embodiment of the invention may comprise an ultra-thin flexible/stretchable battery or comprise a paper-type battery or coin battery. Such a battery may be a primary or secondary battery. In some cases, the power supply unit 140 may comprise an element for performing charging by condensing an inductive current generated by an external magnetic field (e.g., a magnetic field applied an external magnetic charging pad (not shown)). Such an element may include a capacitor and a coil, as illustratively shown in FIG. 1. However, instead of the coil, the conductive grid as described above may be used for generation of an induced current.

According to one embodiment of the invention, the power supply unit 140 may determine whether to supply power or not as the skin attachment 100 is bent. For example, the power supply unit 140 may be configured to supply power once the skin attachment 100 is bent and not to supply power otherwise. To this end, the power supply unit 140 may include a bending sensor (not shown) for sensing bending at a central portion or other suitable location of the skin attachment 100. According to another embodiment of the invention, the power supply unit 140 may supply power only when a certain level or higher of pressure has been applied to the skin attachment 100. To this end, the power supply unit 140 may further include a pressure sensing means (not shown).

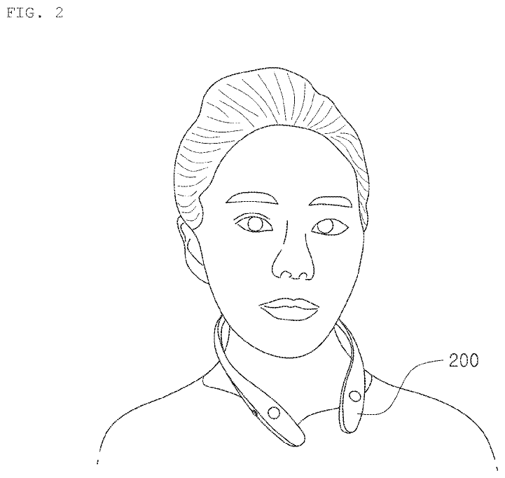

Alternatively, the power supply unit 140 may be electrically wired to a neck band 200 that the user may easily carry around his/her neck (see FIG. 2 in which the user wearing a neck band according to one embodiment of the invention is shown) through the microwire 150 to be described below. The neck band 200 may be separately charged according to a conventional technique to function as a power source. Thus, the user may make movements or perform activities with the skin attachment 100 being attached to his/her face or other body part.

Although the neck band 200 is illustrated above as a power source external to the skin attachment 100, the form of the power source may vary without limitation. For example, the power source may be in the form of a hair band or hairpin that is more natural for a woman to wear. Further, the power source may be implemented in the form of a hat, earphones, headphones, various types of ornaments, a smart watch, or smart glasses, for example.

Meanwhile, the neck band 200 or the like may perform at least part of the above-described functions of the control unit 120.

Further, it is also possible to utilize electricity generated by the user's own body heat or movements, not relying on the power supply unit 140 or relying only partly on the power supply unit 140. For example, it is possible to utilize electricity generated from a plurality of thermoelectric or piezoelectric elements (not shown) that may be disposed on the surface of or inside of the attachment unit 110. Particularly, the plurality of piezoelectric elements may be more useful when the user's body part to which the skin attachment 100 is attached makes a lot of movements or changes its form frequently. The power of the electricity generated as above may be transmitted to the power supply unit 140, but may also be directly transmitted to the light emitting unit 130 and the like.

Lastly, at least a part of the microwire 150 according to one embodiment of the invention may be disposed on the surface of or inside of the attachment unit 110 to achieve the electrical connection as described above. The microwire 150 may have a zigzag shape so that it may be well folded and bended when the skin attachment 100 is of a considerably large size (e.g., when the skin attachment 100 is a sheet mask).

Meanwhile, although the microwire 150 has been described above, the electrical connection may be achieved by a stack of conductors without the microwire, according to one embodiment of the invention.

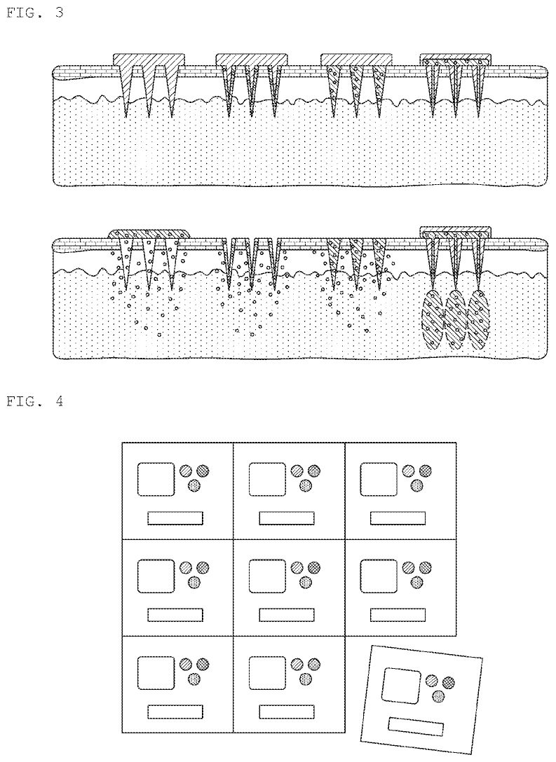

FIG. 3 conceptually shows various types of microneedles. As shown, there are four types of conventional microneedles, i.e., a type of microneedle for making a channel for injecting medicinal substances or the like into skin (shown in the leftmost part); a type of microneedle on which medicinal substances or the like are coated (shown in the next part); a type of microneedle made of a substance that may be dissolved and absorbed (shown in the next part); and a type of microneedle having a hollow therein to allow medicinal substances or the like to be injected through the hollow when the needle penetrates into skin (shown in the rightmost part). Of course, the types may be further subdivided.

According to one embodiment of the invention, the skin attachment 100 may further include or may be coupled to one or more types of microneedles. The microneedles may function to penetrate into the body of the user causing a minimal pain and allow cosmetic or medicinal substances of the attachment unit 110 to be well injected into the skin.

According to one embodiment of the invention, the skin attachment 100 may further comprise a vibration unit (not shown). That is, one or more micro-vibrators or ultrasonic generators (not shown) may be included in the attachment unit 110. Such an element constituting the vibration unit may apply fine vibration to skin of the user according to the control or the like of the control unit 120 and the power supply of the power supply unit 140. The vibration unit may perform a cleansing action to remove waste matter in the user's skin, and may help blood circulation in or around the skin or improve aging of the skin.

Various possible configurations of the skin attachment 100 according to one embodiment of the invention have been described above. Hereinafter, aspects in which the skin attachment 100 is used by the user or applications in which the skin. attachment 100 is used in conjunction with an external device will be described. Further, examples in which the skin attachment 100 comprises additional components will be described. It is to be understood that according to the respective aspects, applications, or additional components, the configuration of the skin attachment 100 may vary widely depending on the judgment of those skilled in the art, although the basic configuration thereof may be maintained to a certain extent.

1. A case where the skin attachment 100 is a patch attached to a narrow region of the user's skin

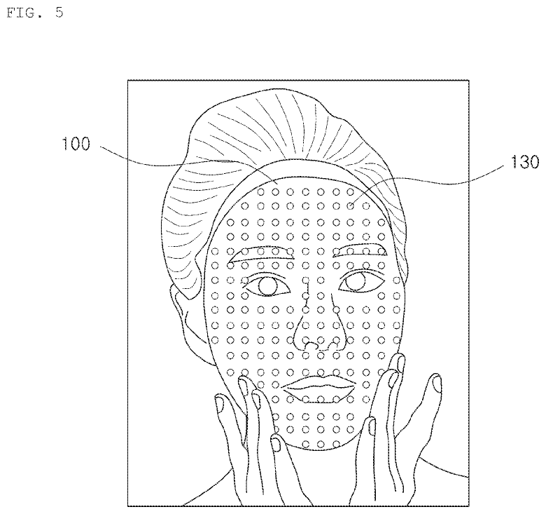

FIG. 4 conceptually shows a collection of skin attachment patches according to one embodiment of the invention. As shown, a plurality of skin attachments 100 may be gathered to form a collection of patches. The user may attach the skin attachment 100, which may be conveniently removed one by one, to a region such as a face requiring skin care.

Due to the small size of the skin attachment 100, it may be preferable to employ a coin battery.

Further, the skin attachment 100 may include a sensor unit as described above. The user may attach the skin attachment 100 including the sensor unit to a region of his/her face or the like for which acquisition. of information on the skin indexes is required (e.g., a region largely affected with acne) to perform phototherapy and make an inspection of the skin. The acquired information may be transmitted to the user's smart phone. Accordingly, the user may retrieve and use quantitative information on the skin condition of a specific region using his/her smart phone. Such quantitative information may include identification information of the corresponding skin attachment 100 (preferably, the identification information may be pre-inscribed on the outside of the skin attachment 100) to assist the user to more intuitively identify a skin region when a plurality of skin regions are inspected using a plurality of skin attachments 100.

2. A case where the skin attachment 100 is a full-size sheet mask covering the user's face

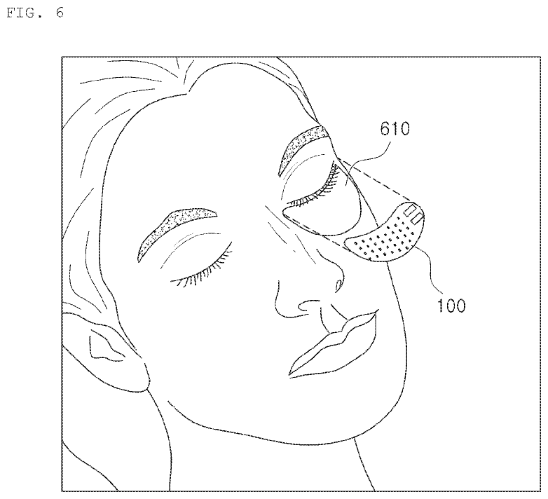

FIG. 5 conceptually shows a skin attachment in the form of a sheet mask according to one embodiment of the invention. As shown, the skin attachment 100 is formed to cover most of the user's face and may include a plurality of distributed light emitting units 130.

Since the size of the skin attachment 100 is relatively large, power may be supplied by the electrolyte or the like permeating in the attachment unit 110.

Further, the skin attachment 100 may also include a sensor unit as described above. The user may acquire information on the skin indexes of each region of his/her face by means of the skin attachment 100 including the sensor unit.

Meanwhile, the phototherapy by the skin attachment 100 may be adjusted according to the feedback control as described above. In the present embodiment, this adjustment may be made more intelligently and effectively because the skin attachment 100 covers the face. For example, with one sheet mask, the user may allow acne treatment to be performed in one region of the face and pain relief to be performed in another region of the face.

3. A case where the skin attachment 100 is used in conjunction with an additional layer 610

FIG. 6 conceptually shows an additional layer that may be coupled to and used in conjunction with a skin attachment according to one embodiment of the invention.

As shown in FIG. 6, before the skin attachment 100 is attached to the user's skin, an additional layer 610 may be first attached in the same region. The skin attachment 100 may then be coupled to the additional layer 610. The additional layer 610 may be made of a material such as a nonwoven fabric, a hydrogel, and a celluloid, and may have a property of transmitting light to some extent. Meanwhile, the skin attachment 100 may include a means for coupling to the additional layer 610 so that it may be easily coupled to the additional layer 610. Such a coupling means may be a Velcro structure on the contact surface of the skin attachment 100.

According to the above configuration, the user may use one skin attachment 100 several times by replacing the disposable additional layer 610 when using the skin attachment 100. Thus, the need to clean the skin attachment 100 may be eliminated or reduced, the cost of the entire product package may be reduced, and the user may have a feeling of more cleanliness. The additional layer 610 may also function to scatter light from the light emitting unit 130 of the skin attachment 100.

4. A case where the skin attachment 100 includes a diffusion unit 710, 720

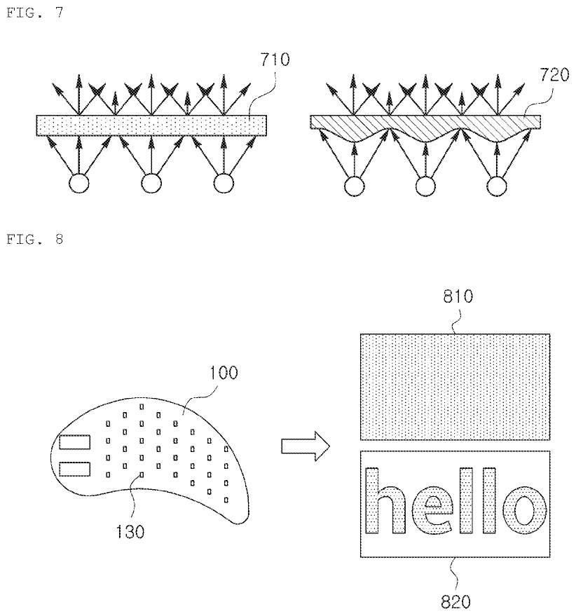

FIG. 7 conceptually shows a diffusion unit that may be included in a skin attachment according to one embodiment of the invention.

As shown in FIG. 7, the skin attachment 100 may include a diffusion unit 710, 720 to cause light from the light emitting unit 130 to be spread and more evenly distributed to the user's skin. As described above, the light emitting unit 130 basically emits light by means of a point light source such as an LED, and thus the intensity of light reaching the use's skin may not be uniform. Accordingly, the diffusion unit 710, 720 may be required.

The diffusion unit 710, 720 may be included in a portion of the skin attachment 100 facing the user (e.g., the attachment unit 110), and the portion may include scattering particles 710 for diffusion of light, or include transparent or translucent microlenses 720. The scattering particles may be bubbles or impurities, and the microlenses may have concave and convex surfaces as shown.

5. A case where the skin attachment 100 provides display through light emitting elements

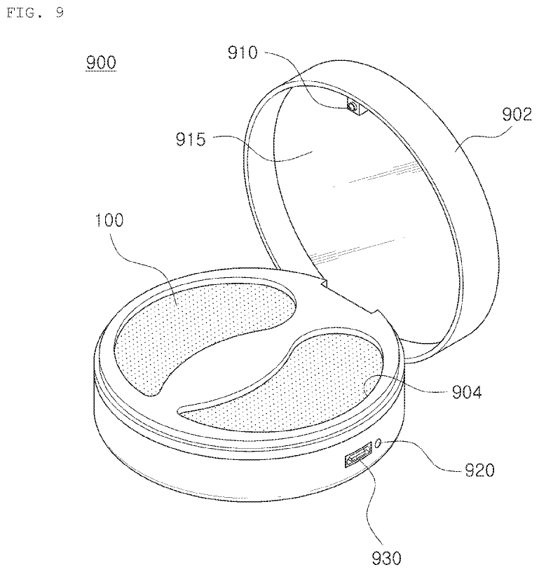

FIG. 8 illustratively shows a situation in which display is performed through light emitting elements of skin attachment according to one embodiment of the invention.

As shown in FIG. 8, at least one light emitting element of the light emitting unit 130 included in the skin attachment 100 may display any type of information on the opposite side of the attachment unit 110. For example, each light emitting element may serve as a single pixel so that information may be displayed in a manner similar to conventional electronic signage, as each pixel emits light or not according to the control of the control unit 120. In FIG. 8, reference numeral 810 denotes a schematic view of the light emitting unit 130 when the light emitting elements do not display any information, and reference numeral 820 denotes a schematic view of the light emitting unit 130 when the light emitting elements display some information (i.e., “hello”).

There is no particular limitation on the type of information that may be displayed. Simple information on the skin attachment 100 such as an operating state (e.g., on/off state) of the skin attachment 100 and a remaining amount of its battery may be displayed, or any characters, pictures, and the like set by the user may be displayed.

### Skin Attachment Storage Apparatus

In the foregoing, the skin attachment 100 according to the invention has been described. Hereinafter, a storage apparatus 900 for storing the skin attachment 100 will be described. The storage apparatus 900 and the skin attachment 100 may form a single product assembly.

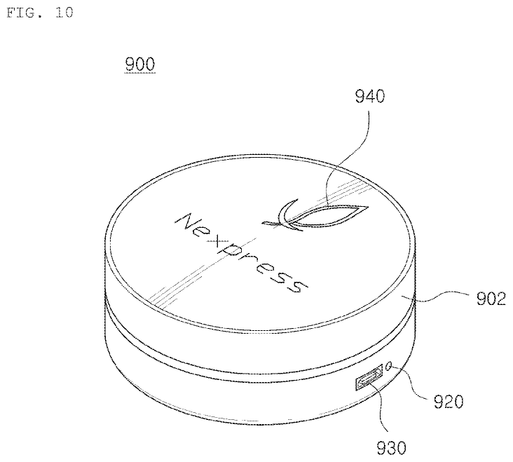

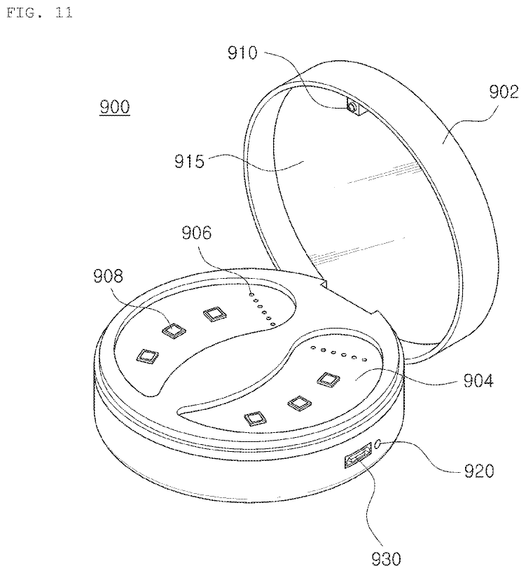

FIGS. 9 to 11 schematically show a skin attachment storage apparatus according to one embodiment of the invention. FIG. 9 shows a situation in which the skin attachment 100 is stored in the storage apparatus 900. FIG. 10 shows a top side of the storage apparatus 900. FIG. 11 shows a situation in which the skin attachment 100 is not stored in the storage apparatus 900.

As shown in FIGS. 9 to 11, the storage apparatus 900 may comprise at least one of a cover 902, a housing unit 904, a charging connection unit 906, a germicidal LED 908, a camera 910, a display 915, a lamp 920, a charging unit 930, and a logo lamp 940. The storage apparatus 900 is digital equipment capable of communication (e.g., WiFi or Bluetooth communication) with the above-described external device as necessary, and any type of digital equipment having a memory means and a microprocessor for computing capabilities may be adopted as the storage apparatus 900 according to the invention. Hereinafter, the respective components of the storage apparatus 900 will be described.

First, the cover 902 may be a component that is coupled to the body of the storage apparatus 900 to allow the storage apparatus 900 to be opened and closed. The camera 910, the display 915, the logo lamp 940, and the like may be disposed therein as will be described below.

Next, the housing unit 904 serves a space for housing and storing the skin attachment 100, and may be open toward the upper side of the storage apparatus 900. The housing unit 904 may contain a liquid for cleaning or disinfecting the skin attachment 100, a solution for beauty care (e.g., essence, lotion, or the like), a therapeutic medicine, and the like, as necessary. Of course, the housing unit 904 may contain the electrolyte as described above.

The inner side of the housing unit 904 may include the charging connection unit 906, which may be connected to the charging unit 930 to be described below. The charging connection unit 906 may be six or a different number of pin connectors. Accordingly, external power may be supplied to the power supply unit 140 of the skin attachment 100 through the charging unit 930 and the charging connection unit 906. Further, through the charging connection unit 906, data for programming the control unit 120 of the skin attachment 100 may be transmitted from the external device to the control unit 120, or measurement values of the sensor unit of the skin attachment 100 may be transmitted to the external device.

A. magnet may be disposed in the charging connection. unit 906 as necessary. In this case, the skin attachment 100 may also include a magnet of opposite polarity at a portion where the microwire 150 connected to the power supply unit 140 is connected to the charging connection unit 906. By the action of these magnets, the microwire 150 of the skin attachment 100 may be connected to the charging connection unit 906 when the user simply places the skin attachment 100 in the housing unit 904.

In addition, the inner side of the housing unit 904 may further include the germicidal LED 908. The skin attachment 100 may be housed and stored in the housing unit 904 as it holds moisture produced in the course of use or cleaning, which increases the likelihood of bacterial growth. Thus, the germicidal LED 908 may be useful.

For example, the germicidal LED 908 may be a UV-C LED that emits ultraviolet light of a wavelength band of 200 to 280 nanometers. This may destroy bacterial DNA and inhibit bacterial growth.

Next, the camera 910 may be a component capable of acquiring an image of an external object (e.g., the user's face). A photograph or video acquired by the camera 910 may be displayed as it is (or as it is processed) by the display 915 to be described below.

Next, the display 915 may show the user a photograph or video of the user's face acquired by the camera 910 before or after the use of the skin attachment 100.

In some cases, the display 915 may communicate with an external device to provide an augmented or virtual image of the user's face, to provide diagnosis/inspection information on the skin condition of the user, and to provide recommendation information on doctors, medicines, beauty products, cosmetics, and the like on the basis of the skin condition of the user. To this end, the storage apparatus 900 may transmit an image of the user's face to an external device and receive feedback thereon from a specific application program of the external device. As an example of such feedback, a mark may be put on a region where acne exists or melanin pigment is largely distributed in the user's face, as the image of the user's face is analyzed.

Next, the lamp 920 may be a component for notifying the user of a charging state of the storage apparatus 900 with a color or flashing state of the lamp 920. The storage apparatus 900 may be charged on its own to act as an external battery for the skin attachment 100 when an external power source is not available.

Next, the charging unit 930 may be a component for allowing power to be supplied to the storage apparatus 900 so that the storage apparatus 900 or the skin attachment 100 therein may be charged. The charging unit 930 may be directly connected to wire that connects with an external AC power source.

The charging unit 930 may be connected to an. electromagnet (not shown) which may be included in the bottom portion of the storage apparatus 900. In this case, the skin attachment 100 may be wirelessly charged by an induced current generated by the electromagnet as described above.

Lastly, the logo lamp 940 may be a component for indicating a charging state of the skin attachment 100. When the charging state of the skin attachment 100 is indicated through the charging connection unit 906, the logo lamp 940 may notify the user of the charging state of the skin attachment 100, rather than the storage apparatus 900, with a color or flashing state of the logo lamp 940.

Although the present invention has been described in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A skin attachment, comprising:
an attachment unit attached to skin of a user, wherein the attachment unit is capable of being bent and attached along a curved surface of the skin of the user;
a light emitting unit disposed in the attachment unit, wherein the light emitting unit comprises at least one light emitting element;
a diffusion unit configured to diffuse light from the light emitting unit, wherein the diffusion unit comprises scattering particles or microlenses; and
a control unit disposed in the attachment unit and configured to control the light emitting unit.

2. The skin attachment of claim 1, wherein a cosmetic or medicinal substance is applied to the attachment unit.

3. The skin attachment of claim 1, wherein the attachment unit includes a capsule in which an ionizing material or electrolyte is encapsulated.

4. The skin attachment of claim 1, wherein the attachment unit includes a conductive grid configured to cause an oxidation-reduction reaction with an ionizing material or electrolyte.

5. The skin attachment of claim 1, wherein each of the at least one light emitting element emits light of a different wavelength band.

6. The skin attachment of claim 1, wherein any one of the at least one light emitting element emits light of any one of a wavelength band of 400 to 430 nanometers, a wavelength band of 630 to 680 nanometers, and a wavelength band of 800 to 900 nanometers.

7. The skin attachment of claim 1, further comprising a sensor unit, wherein the light emitting unit is controlled according to measurement values of the sensor unit.

8. The skin attachment of claim 7, wherein the sensor unit is configured to measure at least one of temperature, pulse, and oxygen saturation in the skin of the user.

9. The skin attachment of claim 7, wherein the sensor unit includes at least one of a gyroscope sensor, an image sensor, a capacitance sensor, and an impedance sensing electrode.

10. The skin attachment of claim 9, wherein the impedance sensing electrode is configured to apply heat.

11. The skin attachment of claim 1, further comprising a vibration unit configured to apply vibration to the skin of the user.

12. The skin attachment of claim 1, wherein the light emitting unit is controlled by the control unit according to communication with an external device.

13. The skin attachment of claim 1, further comprising a coupling means for coupling to an additional layer.

14. The skin attachment of claim 13, wherein the coupling means is a Velcro structure.

15. The skin attachment of claim 1, wherein the at least one light emitting element is a plurality of light emitting elements, and the plurality of light emitting elements are configured to display information on the opposite side of the attachment unit.

16. The skin attachment of claim 1, further comprising a power supply unit configured to supply power to the light emitting unit, wherein the power supply unit includes a bending sensor or pressure sensing means and supplies power when the skin attachment is bent or pressure is applied to the skin attachment.

17. An assembly comprising a skin attachment and a storage apparatus,
wherein the skin attachment comprises:
an attachment unit attached to skin of a user, wherein the attachment unit is capable of being bent and attached along a curved surface of the skin of the user;
a light emitting unit disposed in the attachment unit, wherein the light emitting unit comprises at least one light emitting element; and
a control unit disposed in the attachment unit and configured to control the light emitting unit, and
wherein the storage apparatus comprises:
a housing unit configured to house the skin attachment; and
a charging unit configured to charge the skin attachment.

18. The assembly of claim 17, wherein the skin attachment is charged in the housing unit as connected to the charging unit.

19. The assembly of claim 17, wherein the storage apparatus further comprises a germicidal LED in the housing unit.

20. The assembly of claim 17, wherein the storage apparatus further comprises a camera and a display, and as image acquired by the camera is provided to the user through the display.

21. The assembly of claim 17, wherein the storage apparatus further comprises a camera and a display, and feedback provided by an external device on the basis of an image acquired by the camera is provided to the user through the display.

22. The assembly of claim 17, wherein the storage apparatus further comprises a logo lamp configured to indicate a charging state of the skin attachment.
